(19) United States
(12) Patent Application Publication
(10) Pub. No.: US 2017/0303869 A1
(43) Pub. Date: Oct. 26, 2017

(54) SUB-VIEWPORT LOCATION, SIZE, SHAPE AND/OR ORIENTATION

(71) Applicant: KONINKLIJKE PHILIPS N.V., EINDHOVEN (NL)

(72) Inventor: Liran GOSHEN, PARDES-HANNA (IL)

(73) Assignee: KONINKLIJKE PHILIPS N.V., Eindhoven (NL)

(21) Appl. No.: 15/520,094

(22) PCT Filed: Oct. 21, 2015
(86) PCT No.: PCT/IB2015/058125

§ 371 Date: Apr. 19, 2017

Related U.S. Application Data
(60) Provisional application No. 62/066,962, filed on Oct. 22, 2014.

(51) Int. Cl. A61B 6/03; A61B 5/055; G06T 7/11; G06F 19/00

(52) U.S. Cl. CPC A61B 6/032; A61B 6/037; G06T 7/11; A61B 5/055; G06F 19/00

(57) ABSTRACT

A method includes visually presenting image data (404) in a main window (402) of a display monitor (120). The image data is processed with a first processing algorithm. The method further includes identifying tissue of interest in the image data displayed in the main window. The method further includes generating, with the processor (124), a sub-viewport (502) for the tissue of interest by determining at least one of: a location of the sub-viewport; a size of the sub-viewport; a shape of the sub-viewport; or an orientation of the sub-viewport. The method further includes visually presenting the sub-viewport over a sub-region of the image data in the main window based on one or more of the location, the size, the shape, or the orientation.

### FIELD OF THE INVENTION

The following generally relates to image visualization and is described with particular application to computed tomography (CT). However, the following is also amenable to other imaging modalities such as magnetic resonance (MR), positron emission tomography (PET), single photon emission tomography (SPECT), and/or other imaging modalities.

### BACKGROUND OF THE INVENTION

A CT scanner generally includes an x-ray tube mounted on a rotatable gantry opposite a detector array across an examination region. The rotatable gantry and hence the x-ray tube rotate around the examination region. The x-ray tube emits radiation that traverses the examination region and is detected by the detector array. The detector array generates and outputs a signal indicative of the detected radiation. The signal is reconstructed to generate image data such as 2D, 3D or 4D image data.

For reading, the clinician has viewed image data using different visualization tools. One such tool includes a sub-viewport that enables the clinician to focus on a structure of interest and select a special visualization setting for it, e.g., window level/width, spectral images, etc. This allows the clinician to view the structure of interest in different perspectives in the sub-viewport while having a ‘conventional’ view of the surrounding structures in the main window. This visualization capability facilitates the reading and localizing the structure of interest within the anatomy captured in an image.

One such tool has a sub-viewport that requires the clinician to adjust, manually, the size and shape (or ratio between the rectangle sides) to visualize the structure of interest. Unfortunately, this can be a time consuming and tedious task. Furthermore, the orientation of this sub-viewport has been static with the sides parallel to the main view axes, limiting the ability of the clinician to view the structure of interest in different perspectives in the sub-viewport.

### SUMMARY OF THE INVENTION

Aspects described herein address the above-referenced problems and others.

In one aspect, a method includes visually presenting image data in a main window of a display monitor. The image data is processed with a first processing algorithm. The method further includes identifying tissue of interest in the image data displayed in the main window. The method further includes generating, with the processor, a sub-viewport for the tissue of interest by determining at least one of: a location of the sub-viewport; a size of the sub-viewport; a shape of the sub-viewport; or an orientation of the sub-viewport. The method further includes visually presenting the sub-viewport over a sub-region of the image data in the main window based on one or more of the location, the size, the shape, or the orientation.

In another aspect, a computing apparatus includes a computer processor that executes instructions stored in computer readable storage medium. This causes the computer processor to visually present image data in a main window of a display monitor. The image data is processed with a first processing algorithm. The computer further identifies tissue of interest in the image data displayed in the main window. The computer further generates a sub-viewport for the tissue of interest by determining at least one of: a location of the sub-viewport; a size of the sub-viewport; a shape of the sub-viewport; or an orientation of the sub-viewport. The computer further visually presents the sub-viewport over a sub-region of the image data in the main window based on one or more of the location, the size, the shape, or the orientation.

In another aspect, a computer readable storage medium encoded with computer readable instructions, which, when executed by a processer, causes the processor to: visually present image data in a main window of a display monitor, wherein the image data is processed with a first processing algorithm; identify tissue of interest in the image data displayed in the main window; generate a sub-viewport for the tissue of interest by determining at least one of: a location of the sub-viewport; a size of the sub-viewport; a shape of the sub-viewport; or an orientation of the sub-viewport; and visually present the sub-viewport over a sub-region of the image data in the main window based on one or more of the location, the size, the shape, or the orientation.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example imaging system with a console that includes a set of visualization instructions.

FIG. 2 schematically illustrates an example imaging system with a computing system that includes the set of visualization instructions.

FIG. 3 schematically illustrates an example of the set of visualization instructions.

FIG. 4 illustrates example of a main window visually displaying image data with indicia identifying tissue of interest.

FIG. 5 illustrates the example of FIG. 4 with a sub-viewport superimposed there over.

FIG. 6 illustrate an example method in accordance with the description herein.

### DETAILED DESCRIPTION OF EMBODIMENTS

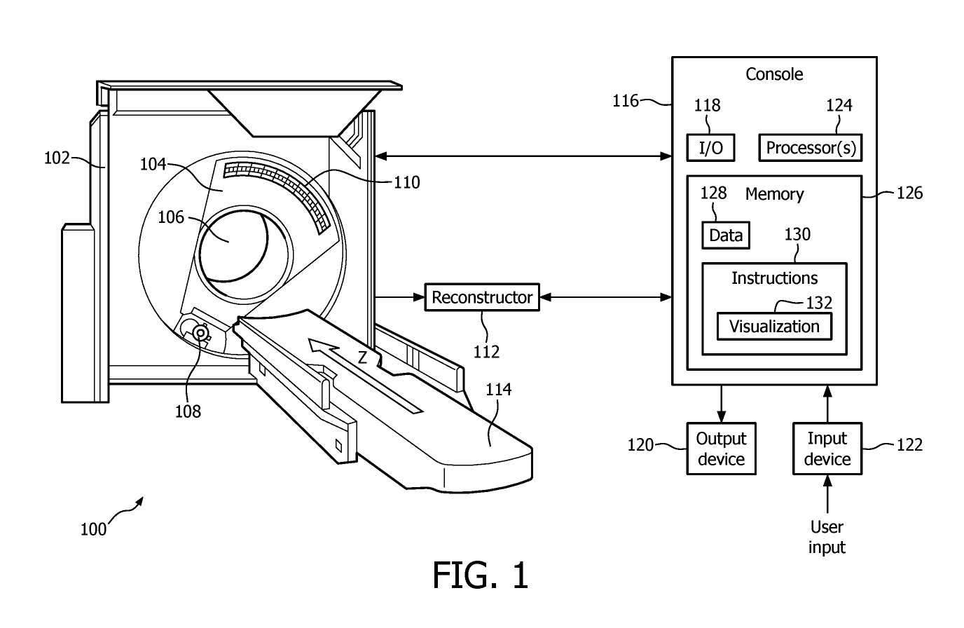

FIG. 1 schematically illustrates an imaging system 100 such as a computed tomography (CT) scanner. The illustrated imaging system 100 includes a generally stationary gantry 102 and a rotating gantry 104. The rotating gantry 104 is rotatably supported by the stationary gantry 102 and rotates around an examination region 106 about a longitudinal or z-axis. A radiation source 108, such as an x-ray tube, is rotatably supported by the rotating gantry 104. The radiation source 108 rotates with the rotating gantry 104 and emits radiation that traverses the examination region 106.

A one-dimensional (1D) or two-dimensional (2D) radiation sensitive detector array 110 subtends an angular arc opposite the radiation source 108 across the examination region 106. The detector array 110 includes one or more rows of detectors arranged with respect to each other along a z-axis direction, detects radiation traversing the examination region 106, and generates signals indicative thereof. A reconstructor 112 reconstructs the signals output by the detector array 110 and generates volumetric image data. A subject support 114, such as a couch, supports an object or subject in the examination region.

A computing system 116 serves as an operator console. The computing system 116 allows an operator to control an operation of the system 100. This includes selecting an imaging acquisition protocol(s), invoking scanning, invoking a visualization software application, interacting with an executing visualization software application, etc. The computing system 116 includes input/output (I/O) 118 that facilitates communication with at least an output device(s) 120 such as a display monitor, a filmer, etc., an input device(s) 122 such as a mouse, keyboard, etc.

The computing system 116 further includes at least one processor 124 (e.g., a central processing unit or CPU, a microprocessor, or the like) and a computer readable storage medium (“memory”) 126 (which excludes transitory medium), such as physical memory and/or other non-transitory memory. The computer readable storage medium 126 stores data 128 and computer readable instructions 130. The at least one processor 124 executes the computer readable instructions 130 and/or computer readable instructions carried by a signal, carrier wave, and other transitory medium.

The computer readable instructions 130 include at least visualization instructions 132. The visualization instructions 132, in one instance, display a main viewport or window that visually presents image data (e.g., 2D, 3D, 4D, etc.) generated using a first algorithm. The visualization instructions 132 further display one or more sub-viewports or sub-windows superimposed over the main viewport. The one or more sub-viewports or sub-windows visually image data (e.g., in 2D, 3D, 4D, etc.), which is under the one or more sub-viewports or sub-windows and in the main view port, using a second or different visualization algorithm.

Examples of the different processing algorithms include, but are not limited to, a poly-energetic X-Ray, a mono-energetic X-Ray, a relative material concentration, an effective atomic number, 2D/3D, and/or other processing algorithm. The other processing can be used to extract additional tissue information, enhance image quality, and/or increase the visualization of tissue/introduced contrast materials. This includes determining clinical values such as the quantification of contrast enhanced tissues, e.g., through an iodine map, generating a virtual non-contrast image from contrast enhanced image data, creating cine mode movies, displaying non-image data through charts, histograms, etc.

As described in greater detail below, the visualization instructions 132, in one instance, automatically sets at least one of a location, a shape, a size or an orientation of the sub-viewport with respect to the image in the main viewport. This may reduce the amount of time it takes to set up the sub-viewport relative to a configuration in which the location, the shape and the size of the sub-viewport are set manually. This also provides further viewing capabilities relative to a configuration in which the orientation of the sub-viewport is static. At least one of the automatically determined location, shape, size or orientation of the sub-viewport can be change, e.g., via the input device 122.

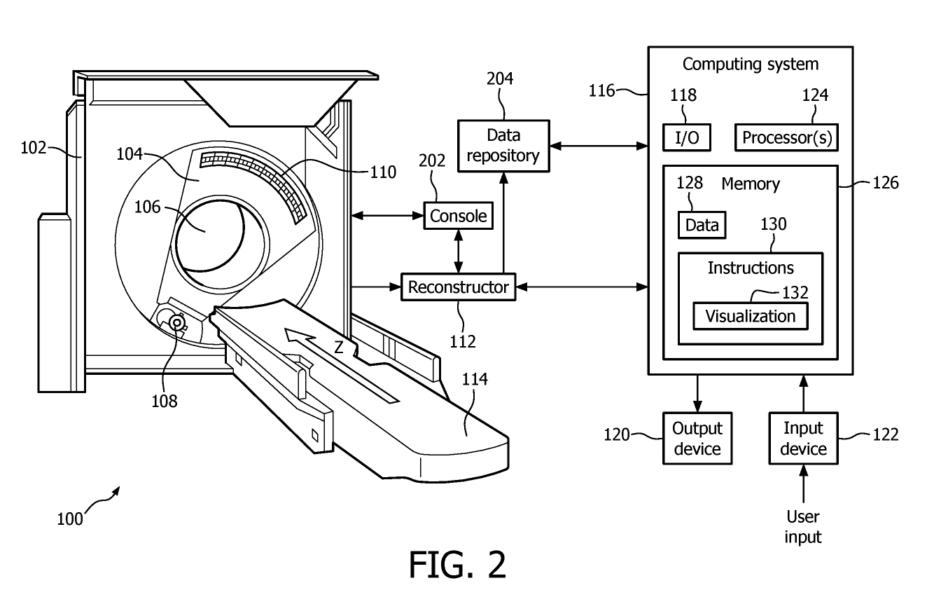

FIG. 2 shows a variation of the system 100 in which the imaging system 100 includes a console 202 and the computing system 116 is separate from the imaging system 100. The computing system 116 obtains the imaging data from the system 100 and/or a data repository 204. An example of a data repository 204 includes a picture archiving and communication system (PACS), a radiology information system (RIS), a hospital information system (HIS), and an electronic medical record (EMR). The imaging data can be conveyed using formats such as Health Level Seven (HL7), Extensible Markup Language (XML), Digital Imaging and Communications in Medicine (DICOM), and/or one or more other format(s).

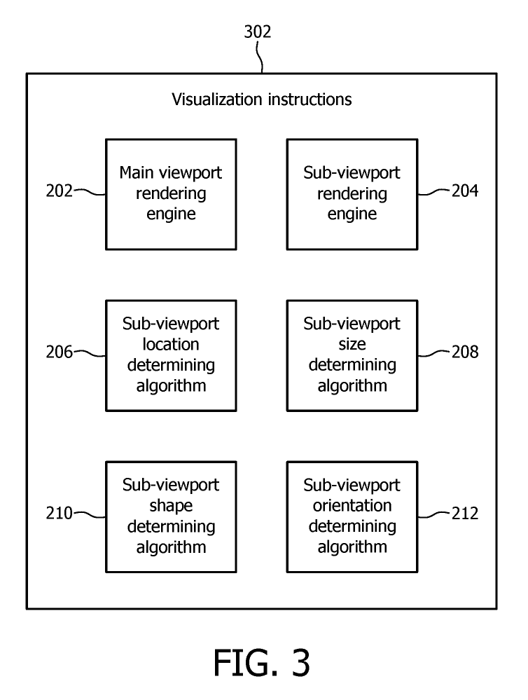

FIG. 3 schematically illustrates an example of the visualization instructions 132.

In this example, the visualization instructions 132 includes a main viewport rendering engine 202, which generates and visually presents a main viewport that visually presents image data processed with a first algorithm. The visualization instructions 132 also include a sub-viewport rendering engine 204, which generates and visually presents a sub-viewport that visually presents a sub-portion of the image data, which is processed with a second or different algorithm, including the region of the image data under the sub-viewport. The sub-viewport can be moved through the imaging data via the input device 122.

The visualization instructions 132 further include a sub-viewport location determining algorithm 206. The processor 124, in response to executing the algorithm 206, determines a location for the sub-viewport within the main viewport. In one instance, this includes receiving an input from the input device 122 indicating a location within the main viewport. For example, the input may be indicative of a point in the main viewport selected via a mouse click. In another instance, this includes automatically determining the location based on processing of the image data. The location can be determined automatically based on an identification of tissue of interest by a computer-aided detection algorithm.

The visualization instructions 132 further include a sub-viewport size determining algorithm 208. The processor 124, in response to executing the algorithm 208, determines a size of the sub-viewport in the main viewport. In one instance, the processor 124 determines a size of the sub-viewport by searching for local extremity (e.g., minima and/or maxima) values across all possible scales, using a continuous function of scale, or a scale space.

The scale space of an image, for example, can be defined in 2D space as a function, L(x, y, σ), that is produced from the convolution of a variable-scale Gaussian, G(x, y, σ), with an input image, I(x, y) as follows: L(x, y, =G(x, y, σ)*I(x, y), where * is a convolution operation in x and y, and

$$G ( x , y , σ ) = \frac{1}{2 {Πσ}^{2}} {e}^{- ( {x}^{2} + {y}_{2} ) / ( 2 {σ}^{2} )} .$$

For instance, to set the size, local extremity values of σ in the space scale L(x, y, σ), where x and y define the location of the sub-viewport, are detected. If several extremities are found, the \hat{σ} that is closest to a predefine value is identified and selected. Then, the size of the sub-viewport is set by a multiple of the selected \hat{σ} by predefined scale factor.

The visualization instructions 132 further include a sub-viewport shape determining algorithm 210. The processor 124, in response to executing the algorithm 210, determines a shape of the sub-viewport. In one instance, this includes setting the shape using a structure tensor. In general, the structure tensor summarizes the predominant directions of the gradient in a specified neighborhood of a point and the degree to which those directions are coherent. The following example is for a rectangular shaped sub-viewport.

For instance, to set the shape of the sub-viewport, the processor 124 scales down the image to the scale determined through the sub-viewport size determining algorithm 208, i.e., the scale corresponding to \hat{σ}. Then, the structure tensor is calculated. Then, the eigenvalues and the corresponding eigenvectors of the structure tensor matrix are calculated. Then, a ratio between the sides of the sub-viewport window is set to be the ratio between the square root of the eigenvalues. The ratio could be cropped by predefined upper threshold and/or lower threshold.

The following is an example calculation, for the discrete case, of the structure tensor at 2D point p=(x,y):

$${S}_{w} [ p ] = [ \begin{matrix}{Σ}_{r} w [ r ] {( {I}_{x} [ p - r ] )}^{2} & {Σ}_{r} w [ r ] {I}_{x} [ p - r ] {I}_{y} [ p - r ] \\ {Σ}_{r} w [ r ] {I}_{x} [ p - r ] {I}_{y} [ p - r ] & {Σ}_{r} w [ r ] {( {I}_{y} [ p - r ] )}^{2}\end{matrix} ] .$$

In the foregoing, the summation index r ranges over a finite set of index pairs (the “window” typically {−m . . . +m}×{−m . . . +m} for some m), and w[R] is a fixed “window weight” that depends on r such that the sum of all weights is one (1).

The following is an example calculation, for the continuous case, of the structure tensor for a function I of three variables p=(x,y,z): S_{w}[p]=∫w[r]S₀(p−r)dr, where

$${S}_{0} [ p ] = [ \begin{matrix}{( {I}_{x} ( p ) )}^{2} & {I}_{x} ( p ) {I}_{y} ( p ) & {I}_{x} ( p ) {I}_{z} ( p ) \\ {I}_{x} ( p ) {I}_{y} ( p ) & {( {I}_{y} ( p ) )}^{2} & {I}_{y} ( p ) {I}_{z} ( p ) \\ {I}_{x} ( p ) {I}_{z} ( p ) & {I}_{y} ( p ) {I}_{z} ( p ) & {( {I}_{z} ( p ) )}^{2}\end{matrix} ] ,$$

where iₓ, i_{y}, I_{z}, are the three partial derivatives of I, and the integral ranges over [symbol] . In the discrete version,

$${S}_{w} [ p ] = {Σ}_{r} w [ r ] {S}_{0} [ p - r ] , where {s}_{0} [ p ] = [ \begin{matrix}{( {I}_{x} ( p ) )}^{2 γ} & {I}_{x} ( p ) {I}_{y} ( p ) & {I}_{x} ( p ) {I}_{z} ( p ) \\ {I}_{x} ( p ) {I}_{y} ( p ) & {( {I}_{y} ( p ) )}^{2} & {I}_{y} ( p ) {I}_{z} ( p ) \\ {I}_{x} ( p ) {I}_{z} ( p ) & {I}_{y} ( p ) {I}_{z} ( p ) & {( {I}_{z} ( p ) )}^{2}\end{matrix} ]$$

and the sum ranges over a finite set of 3D indices, e.g., {−m . . . +m}×{−m . . . +m}×{−m . . . +m} for some m.

Adding an additional dimension to the matrix, e.g., for the additional dimension t, an additional row and column, related to the additional dimension t and its derivative Iₜ, are added to the matrix:

$$[ \begin{matrix}{( {I}_{x} ( p ) )}^{2} & … & {I}_{x} ( p ) {I}_{t} ( p ) \\ ⋮ & ⋱ & ⋮ \\ {I}_{x} ( p ) {I}_{t} ( p ) & … & {( {I}_{t} ( p ) )}^{2}\end{matrix} ] .$$

The visualization instructions 132 further include a sub-viewport orientation determining algorithm 212. The processor 124, in response to executing the algorithm 212, determines a spatial orientation of the sub-viewport in the main viewport. In one instance, this includes setting the orientation of a major side of the sub-viewport window to be an orientation of the eigenvector that corresponds to a smallest eigenvalue of the structure tensor.

The following example is for an elliptical shaped sub-viewport. An elliptical shaped sub-viewport can be defined by its semi-major axis and its semi-minor axis. In one instance, this includes setting a length of the semi-major axis by multiplying the selected \hat{σ} with a predefined scale factor, which can be predetermined, specified by a user, etc. A length of the semi-minor axis is set by multiplying the semi-major axis length by a ratio between the square root of the eigenvalues of the structure tensor. The orientation of the semi-major axis is set to be the orientation of the Eigen vector that is corresponding to the smallest Eigen value of the structure tensor. The orientation of the semi-minor axis is perpendicular to the semi-major axis.

Note that the user could drag the sub-viewport through the image/dataset and the sub-viewport could change its size, shape and orientation on the fly according to the current location. The proposed algorithm improves the usability of the sub-viewport by automatically setting the shape, size and even the orientation of the sub-viewport. The algorithm could also be used to set a viewport in 4D and/or dynamic contract enhanced cases. In this instance, the size, shape and/or orientation can be dynamically adjusted based on movement of surrounding structure. In addition, the sub-viewport could have other shapes.

Furthermore, a toggle feature allows a user to toggle sub-viewport on and off. The toggle feature can be activated, for example, via a signal from the input device 122 indicative of a user selecting the toggle feature. When on, the sub-viewport is visible over the image in the main window. When off, the sub-viewport is not visible over the image in the main window. When off, the sub-viewport may not be overlaid over the image in the main window or it may be overload over the image in the main window, but transparent. For example, in one instance, in response to a toggle signal indicating the sub-viewport should be removed, the visual presentation of the sub-viewport is removed from the main window. In another example, in response to a toggle signal indicating the sub-viewport should be hidden, the sub-viewport is hidden, for example, rendered transparent or otherwise made invisible to the human observer.

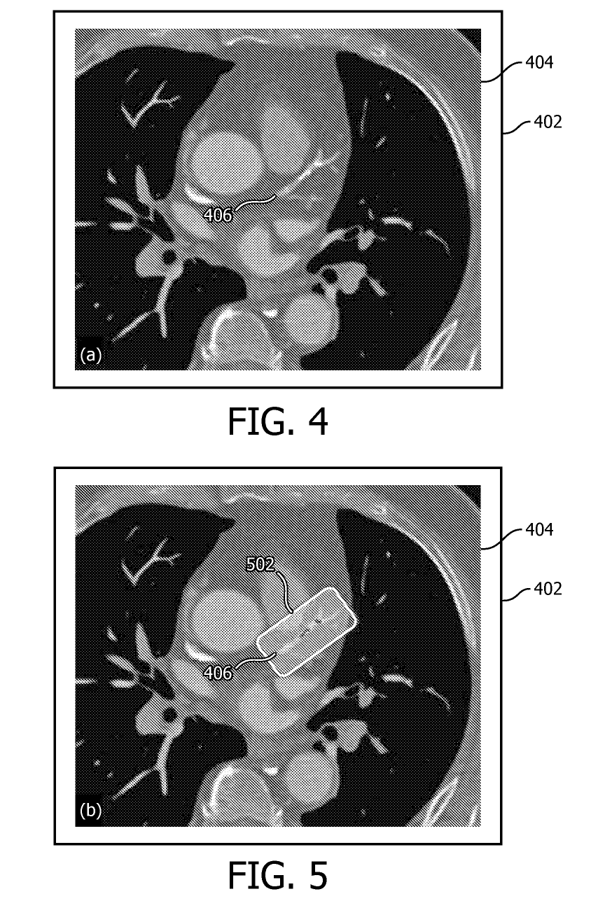

FIG. 4 illustrates example of a main window 402 visually displaying cardiac image data 404. Indicia 406 identifies tissue of interest automatically selected by a processor executing software and/or manually selected through in input signal indicative of a user selection. In this example, the tissue of interest includes the left anterior descending (LAD) coronary artery.

FIG. 5 illustrates the main window 402 displaying the cardiac image data 404 with a sub-viewport 502 superimposed there over. In this example, the sub-viewport 502 location, size, shape and/or orientation corresponds to the tissue of interest identified by the indicia 406 such that the sub-viewport 502 is located over the tissue of interest and displays the same tissue located underneath the sub-viewport 502 but processed with a second different processing algorithm. In this example, the sub-viewport window 502 visually displays a color-coded map of spectral effective atomic number map.

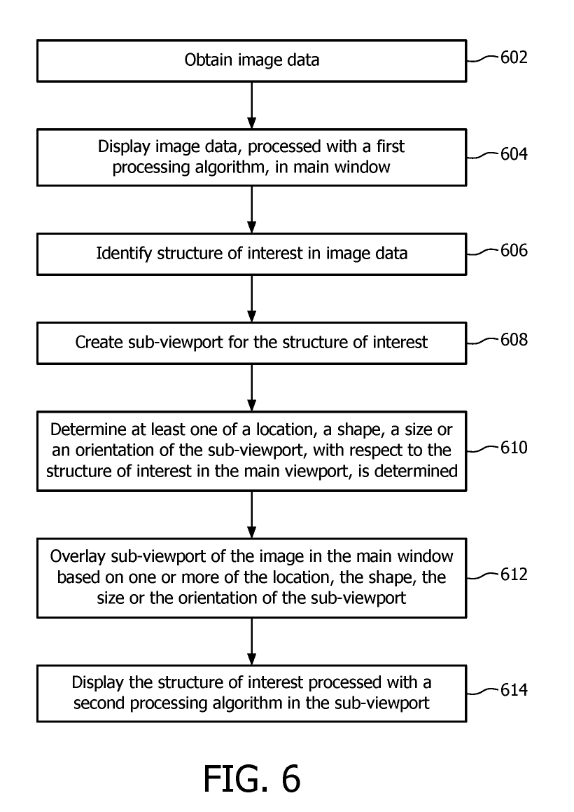

FIG. 6 illustrate an example method.

It is to be appreciated that the ordering of the acts in the method is not limiting. As such, other orderings are contemplated herein. In addition, one or more acts may be omitted and/or one or more additional acts may be included.

At 602, image data, created by processing projection and/or image data with a first processing algorithm, is obtained.

At 604, the image data is visually displayed in a main window of a GUI visually presented via a display monitor.

At 606, a structure of interest is identified in the image data.

At 608, a sub-viewport is created for the structure of interest.

At 610, at least one of a location, a shape, a size or an orientation of the sub-viewport, with respect to the structure of interest in the main viewport, is determined.

At 612, the sub-viewport is overlaid over the image in the main window based on at least one of the determined location, the shape, the size or the orientation.

At 614, the structure of interest in the sub-viewport is processed with a second different processing algorithm.

A toggle feature allows a user to toggle sub-viewport on and off. When on, the sub-viewport is visible over the image in the main window. When off, the sub-viewport is not visible over the image in the main window. When off, the sub-viewport may not be overlaid over the image in the main window or it may be overload over the image in the main window, but transparent.

The above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising:
visually presenting image data in a main window of a display monitor, wherein the image data is processed with a first processing algorithm;
identifying, with a processor, tissue of interest in the image data displayed in the main window;
generating, with the processor, a sub-viewport for the tissue of interest by determining at least one of:
a location of the sub-viewport;
a size of the sub-viewport;
a shape of the sub-viewport; or
an orientation of the sub-viewport; and
visually presenting, with the processor, the sub-viewport over a sub-region of the image data in the main window based on one or more of the location, the size, the shape, or the orientation.

2. The method of claim 1, further comprising:
receiving a first input indicating the tissue of interest in the image data, wherein the first input is indicative of a user selected tissue of interest; and
determining the location of the sub-viewport based on the first input.

3. The method of claim 1, further comprising:
receiving a first input indicating the tissue of interest in the image data, wherein the first input is indicative of a processor selected tissue of interest; and
determining the location of the sub-viewport based on the first input.

4. The method of claim 1, wherein determining the size of the sub-viewport comprises: determining scale spaces of the image data; searching for local minima and maxima values of the tissue of interest across the scale spaces; identifying a local minima and a local maxima for a scale space; and multiplying the local minima and the local maxima by a predefined scale factor.

5. The method of claim 4, wherein a scale space is determined by convolving a variable-scale Gaussian function with the image data.

6. The method of claim 1, wherein determining the shape of the sub-viewport comprises: scaling down the image data to the scale of the local minima and the local maxima; calculating a structure tensor which identifies predominant directions of a gradient in a specified neighborhood of a point and a degree to which those directions are coherent; calculating an eigenvalues and corresponding eigenvectors of the structure tensor matrix; and setting a ratio between sides of the sub-viewport to a ratio between a square root of the eigenvalues.

7. The method of claim 6, further comprising:
cropping the ratio by at least one of a predefined upper threshold or a predefined lower threshold.

8. The method of claim 6, wherein determining the orientation of the sub-viewport comprises: setting the orientation of a major side of the sub-viewport to be the orientation of the eigenvector corresponding to a smallest eigenvalue of the structure tensor.

9. The method of claim 1, further comprising:
receiving a signal indicating movement of the sub-viewport through the image data; and
updating, with the processor, at least one of the location, the size, the shape, or the orientation of the sub-viewport based on the structure of interest at the location of the sub-viewport in the image data.

10. The method of claim 1, further comprising:
receiving a toggle signal to remove the sub-viewport; and
removing the visual presentation of the sub-viewport from the main window.

11. The method of claim 1, further comprising:
receiving a toggle signal to hide the sub-viewport; and
rendering the sub-viewport transparent.

12. The method of claim 1, wherein the image data is one of a 2D image, 3D volumetric image data or 4D image data.

13. The method of claim 12, further comprising:
dynamically adjusting at least one of the location, the size, the shape and the orientation of the sub-viewport based on movement of surrounding structure.

14. A computing system, comprising:
a computer processor configured to execute instructions stored in computer readable storage medium which causes the computer processor to:
visually present image data in a main window of a display monitor, wherein the image data is processed with a first processing algorithm;
identify tissue of interest in the image data displayed in the main window;
generate a sub-viewport for the tissue of interest by determining at least one of: a location of the sub-viewport; a size of the sub-viewport; a shape of the sub-viewport; or an orientation of the sub-viewport; and
visually present the sub-viewport over a sub-region of the image data in the main window based on one or more of the location, the size, the shape, or the orientation.

15. The computing system of claim 14, wherein the processor determines the size of the sub-viewport by determining scale spaces of the image data; searching for local minima and maxima values of the tissue of interest across the scale spaces; identifying a local minima and a local maxima for a scale space; and multiplying the local minima and the local maxima by a predefined scale factor.

16. The computing system of claim 15, wherein the processor determines the shape of the sub-viewport by scaling down the image data to the scale of the local minima and the local maxima; calculating a structure tensor which identifies predominant directions of a gradient in a specified neighborhood of a point and a degree to which those directions are coherent; calculating an eigenvalues and corresponding eigenvectors of the structure tensor matrix; and setting a ratio between sides of the sub-viewport to a ratio between a square root of the eigenvalues.

17. The computing system of claim 16, wherein the image data is one of a 2D image, 3D volumetric image data or 4D image data.

18. The computing system of claim 14, wherein the computing system is part of a console of an imaging system.

19. The computing system of claim 14, wherein the computing system is an apparatus separate and remote from an imaging system.

20. A computer readable storage medium encoded with one or more computer executable instructions, which, when executed by a processor of a computing system, causes the processor to:
visually present image data in a main window of a display monitor wherein the image data is processed with a first processing algorithm;
identify tissue of interest in the image data displayed in the main window;
generate a sub-viewport for the tissue of interest by determining at least one of: a location of the sub-viewport; a size of the sub-viewport; a shape of the sub-viewport; or an orientation of the sub-viewport; and
visually present the sub-viewport over a sub-region of the image data in the main window based on one or more of the location, the size, the shape, or the orientation.
